(19) United States
(12) Patent Application Publication
(10) Pub. No.: US 2013/0125316 A1
(43) Pub. Date: May 23, 2013

(54) System and a Method for Washing, Cleaning, Disinfecting and Sanitizing Laundry Using Electrolytic Cell Having Boron-Doped Diamond Electrode

(71) Applicants: Haresh Jitendrarai Bhuta, Gujarat (IN); Nirjar Rajendra Bhatt, Gujarat (IN)

(72) Inventors: Haresh Jitendrarai Bhuta, Gujarat (IN); Nirjar Rajendra Bhatt, Gujarat (IN)

(73) Assignee: XH20 SOLUTIONS PRIVATE LIMITED, Ahmedabad (IN)

(21) Appl. No.: 13/813,556

(22) PCT Filed: Jul. 7, 2011
(86) PCT No.: PCT/IN11/00456

§ 371 Date: Jan. 31, 2013

(30) Foreign Application Priority Data
Aug. 2, 2010 (IN) 2190/MUM/201

(51) Int. Cl. C25F 1/00

(52) U.S. Cl. CPC C25F 1/00
USPC 8/137; 204/240

(57) ABSTRACT

The invention relates to a system and a method for washing, cleaning, disinfecting and sanitizing laundry using electrolyte solution containing mixed oxidants generated in-situ using electrochemical reaction. More particularly the present invention relates to a system and a method for washing, cleaning, disinfecting and sanitizing laundry at a pH between 6.5 to 10.5 and at a temperature below 50° C. using in-situ mixed oxidants generated by passing electrolyte solution through electrolytic cell having boron-doped diamond electrode.

### FIELD OF THE PRESENT INVENTION

The invention relates to a system and a method for washing, cleaning, disinfecting and sanitizing laundry using electrolyte solution containing mixed oxidants generated in-situ using electrochemical reaction. More particularly the present invention relates to a system and a method for washing, cleaning, disinfecting and sanitizing laundry at a pH between 6.5 to 10.5 and at a temperature below 50° C. using in-situ mixed oxidants generated by passing electrolyte solution through at least one electrolytic cell having at least one Boron-doped diamond electrode.

### BACKGROUND OF THE PRESENT INVENTION

Conventional methods for washing, cleaning, disinfecting and sanitizing laundry such as but not limited to linen, industrial uniforms, towels, napkins, hospitals, or any other textile or laundry is carried out at a higher temperature of more than 50° C. using detergents, enzymes, detergent with enzymes, disinfectants natural or synthetic, bleaching agents like hypochlorite (NaOCl), hydrogen peroxide (H₂O₂). These present washing, cleaning and disinfecting practice uses energy, water and chemicals to achieve the desired result. The present methods and protocols have been in practice since decades and gets more and more severe on resource consumption each day, as the desired washing, cleaning, disinfecting and sanitizing becomes more difficult. The end result is that economic and environmental cost of the cleaning/washing services for linen/related objects is on a rise.

There are several prior arts reported for electrochemical reaction using different type of electrodes or electrolytic cell.

U.S. Pat. No. 5,399,247 dated Dec. 22, 1993 of Eastman Kodak Company has disclosed a method for the treatment of the waste water or solution wherein a solute is treated in a liquid solution in order to render the solution more acceptable for discharge into an environment. According to the method, the solute was oxidized by electrolyzing the solution with an anode wherein anode comprises the doped diamond in the form of a layer on an electrically conductive substrate like molybdenum, tungsten, titanium, copper, cobalt, chromium, nickel, tantalum, zirconium, niobium, doped silicon and a suboxide of titanium. The solution which is electrolyzed using an anode is selected from industrial waste or photo processing solution. The patent does not teach about the system and a method for washing, cleaning and disinfecting laundry.

U.S. 2009/032409 dated Feb. 12, 2008 of Juan Horn has disclosed a process for cleaning, sterilizing and disinfecting dishes and other kitchen utensils by means of a wash liquid wherein, by direct application of electrical current to a diamond and/or lead-tin electrode arranged in the wash liquid, OH radicals are generated in the wash liquid which radical effect the cleaning, sterilization and disinfection of the dishes and other kitchen utensils.

WO2009/067838 dated Nov. 26, 2008 has disclosed a method and technical design for cleaning laundry, crockery, vehicles and floor surfaces with electrolyzed water by means of oxidative radicals produced by diamond electrodes. The invention relates to a method and a device for the non-chemical, residue-free cleaning, sanitization, disinfection and odor neutralization of laundry, textiles, crockery, floor surfaces, vehicles and animals, in addition to surfaces, materials and objects, by means of electrolytic washing and cleaning technology and an electrolysis generator comprising diamond electrodes doped with boron or with other atoms, using electrolyzed hot or cold water by means of oxidative radicals.

U.S.2007/0180866 of Haier Group Corporation has disclosed a washing machine and a method for washing of laundry using electrolysis cell with diaphragm wherein according to the method, modifying agents alkyl polyglycoside or citric acid trialkylamide are used at a pH between 8.5 to 11 and at a temperature between 5° C. to 50° C.

U.S. Pat. No. 6,387,241 dated Oct. 25, 1999 of Lyntech has disclosed a method for washing laundry wherein laundry is placed in a vessel and filled the vessel with water. Ozone gas is passed in sealed vessel to allow the ozone to clean the laundry. Ozone is produced under air pressure by an electrochemical cell which comprises; an anode comprises a substrate and a catalyst coating, wherein substrate is selected from the group consisting of porous titanium, titanium suboxides, tantalum, hafnium and niobium and wherein the catalyst coating is selected from group consisting of lead dioxide, boron-doped diamond etc and a cathode and proton exchange membrane.

The advantages of electrochemical reaction using anodes over chemical and thermal processes are the ease of operation, simplicity of design, and relatively small equipment space requirements. Electrolysis is also considered to be relatively safe to operate when compared to oxidative treatment techniques which necessitate handling powerful chemical oxidants.

However, there are several problems with the selection of type of anode while electrochemical reaction. Most anode materials like platinum, ruthenium dioxide, lead oxide, and tin oxide lead to discharge toxic materials into the environment. Other novel mixed metal oxide electrode coating or electrodes including graphite and carbon electrodes either solid/felt/nano tubes are not efficient in generating oxidants at electrode surface within their operating limits of current density. Most of the anode materials are reported as less effective in electrochemical reaction which amounts into the requirement of lengthy time to complete the electrochemical reaction and relatively high amounts of energy to achieve desire results which ultimately results into the expensive process for electrochemical reaction.

Further it is known that at the time of electrochemical reaction using certain anodes results into the side reactions and thus undesirable impurities and results found during the process which ultimately results into the less efficient and costly process.

Another disadvantage is that as suggested in the above prior art, during the electrochemical reaction, disinfecting and cleaning agent Ozone (O₃) is supplied externally through commercially available sources. The utmost care must be taken for externally supplied ozone as there may be problems like storage of the cylinders, leakage of Ozone gas at the time of passing into the system. Leakage of Ozone leads to the problems like non-achievement of the desired results, higher volume of ozone gas required for the completion of electrochemical reaction process thus makes the process and a system industrially not applicable due to lower efficiency, non-achievement of the desired results and input of the higher cost for the efficiency of the process.

Furthermore, there is no prior art suggested for a system and a method for washing, cleaning, disinfecting and sanitizing laundry by electrochemical reaction at a pH between 6.5 to 10.5 and at a temperature below 50° C. using in-situ mixed oxidants generated by passing electrolyte solution through electrolytic cell having boron-doped diamond electrode.

There is a huge gap and demand in the field of commercial laundry, hotels, hospitals, nursing homes, prisons, armed forces, textile and in residential part for soapless, chemical less, easy to operate, environment friendly, harmless, industry-viable and a cost-effective system and a method for washing, cleaning, disinfecting and sanitizing laundry.

Therefore, there is an urgent need for a system and a method for washing, cleaning, disinfecting and sanitizing laundry using in-situ mixed oxidants generated by electrochemical reaction at a pH between 6.5 to 10.5 and at a temperature below 50° C. by passing electrolyte solution through electrolytic cell having at least one Boron-doped diamond electrode that will avoid or minimize the problems and drawbacks described above.

### SUMMARY OF THE PRESENT INVENTION

The present invention meets the above noted need by providing a system and a method for washing, cleaning, disinfecting and sanitizing laundry by electrochemical reaction.

More particularly the present invention provides a system and a method for washing, cleaning, disinfecting and sanitizing laundry using in-situ mixed oxidants generated by electrochemical reaction at a pH between 6.5 to 10.5 and at a temperature below 50° C. by passing electrolyte solution through electrolytic cell having at least one boron-doped diamond electrode.

The invention enables not only cleaning, but also disinfect and sanitize the laundry and is able to eliminate micro-organisms such as viruses, gram-positive and gram-negative bacteria, etc., by means of electrochemical reaction at a temperature below 50° C., without using washing agents or disinfectants.

It has been observed and measured by various studies and experiments that during electrochemical reaction of electrolyte solution using boron-doped diamond electrode in presence of direct current produces Hydroxyl (OH) radicals which leads to the formation of mixed oxidants like ozone, hypochlorite, percarbonate, persulfate, hydrogen peroxide in electrolyte solution. Experiments for washing, cleaning, disinfecting and sanitizing laundry have proved that mixed oxidants possess unique washing, cleaning, disinfecting and sanitizing properties on soiled linen or laundry. Experiments have also proved that in-situ generated mixed oxidants have better washing, cleaning, disinfecting and sanitizing efficiency than the methods and a system suggested in the prior art.

Therefore, the main object of the present invention is to provide soapless, chemical less, easy to operate, environment friendly, harmless, industry-viable and a cost-effective system and a method for washing, cleaning, disinfecting and sanitizing laundry.

There is further an object to provide a system and a method for washing, cleaning, disinfecting and sanitizing laundry using in-situ mixed oxidants generated by electrochemical reaction at a pH between 6.5 to 10.5 and at a temperature below 50° C. by passing electrolyte solution through at least one electrolytic cell having at least one boron-doped diamond electrode.

It is also an object to provide a system and a method for washing, cleaning, disinfecting and sanitizing laundry wherein the electrolyte solution for the purpose of the electrochemical reaction process is prepared according to the purpose and needs of the application for cleaning, disinfecting and sanitizing laundry.

### BRIEF DESCRIPTION OF DRAWINGS

For a fuller understanding of the nature and objects of the present invention, reference should be made to the following detailed description, taken in connection with the following drawings without limiting the scope of the invention, in which:

FIG. 1 is a schematic diagram of a system for washing, cleaning, disinfecting and sanitizing laundry

FIG. 2 is a schematic diagram of a system for washing, cleaning, disinfecting and sanitizing laundry for more than one washing apparatus FIG. 3 is a schematic diagram of a system for washing, cleaning, disinfecting and sanitizing laundry along with a system of recycling and reuse of electrolyte solution containing mixed oxidants

FIG. 4 is of wash results of example-1 obtained using a system and a method of the present invention

FIG. 4A Cloth having stain of soya sauce before washing

FIG. 4B Cloth having stain of soya sauce after washing

FIG. 4C Cloth having stain of chocolate sauce before washing

FIG. 4D Cloth having stain of chocolate sauce after washing

FIG. 5 is of wash results of example-2 obtained using a system and a method of the present invention

FIG. 5A Cloth having stain of soya sauce before washing

FIG. 5B Cloth having satin of soya sauce after first washing

FIG. 5C Cloth having salad dressing before washing

FIG. 5D Cloth having salad dressing after first washing

FIG. 6 is of disinfection and sanitization results obtained using a system and a method of the present invention

FIG. 6A Before wash, Initial CFU: 10⁶, Specimen used Cloth

FIG. 6B After wash, Treatment time: 10 minutes, Final CFU: 0

### DETAILED DESCRIPTION OF THE PRESENT INVENTION

The present invention relates to a system and a method for washing, cleaning, disinfecting and sanitizing laundry at a pH between 6.5 to 10.5 and at a temperature below 50° C. by means of passing the electrolyte solution through electrolytic cell having at least one boron-doped diamond electrode in presence of direct current (DC). OH radicals are generated in the electrolyte solution which further leads to generate mixed oxidants such as ozone, hypochlorite, persulfate, percarbonate and hydrogen peroxide into the electrolyte solution which have a better washing, cleaning efficiency than the prior art. Moreover, a cell-killing effect can be achieved with the mixed oxidants in relation to microorganisms, germs and bacteria.

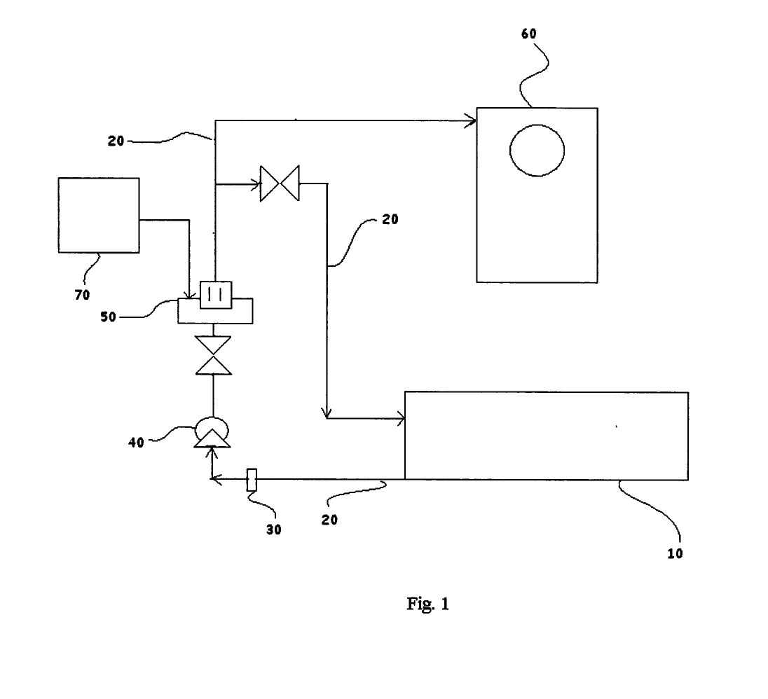

Referring to FIG. 1, there is a system disclosed for washing, cleaning and disinfecting laundry which comprises:
at least one electrolyte solution reservoir;
at least one filter;
at least one pump;
at least one electrolytic cell having at least one boron-doped diamond electrode;
at least one washing apparatus;
at least one means for providing DC power; and
a plurality of conduits for conveying electrolyte solution.

Reservoir (10) is used for the purpose of preparing, storing and conveying electrolyte solution. At least one reservoir is required for the purpose of preparing, storing and conveying electrolyte solution.

Optionally at least one filter (30) in a system is provided to block unwanted particles from the electrolyte solution. Arrangement of the filter may be possible in two ways: (i) Filter can be provided in between electrolyte solution reservoir (10) and pump (40) or (ii) Filter can be provided between pump and electrolytic cell. Further electrolyte solution reservoir (10), Filter (30) and pump (40) are connected through conduit for conveying electrolyte solution (20). Filter (30) of the said system can be selected from the range of the filters which are commercially available but the preferred filter to block the unwanted particles and further to get the desired results would be of less than 50 microns.

At least one electrolytic cell (50) having at least one boron-doped diamond electrode is connected with the system through conduit for conveying electrolytic solution (20). Boron-doped diamond electrode in plurality can also be used according to the application of washing, cleaning, disinfecting and sanitizing laundry. Boron-doped diamond electrodes exhibit interesting electrochemical properties, which include a wide potential window, stability in electrolyte solution, low background current density and high resistance against chemical and electrochemical corrosion. These make the boron-doped diamond electrode a preferred electrode for various electrochemical applications. According to the disclosure of the patents U.S. Pat. No. 5,399,247, U.S. 20060261349 and U.S. Pat. No. 7,407,566 and disclosure in the prior art “Electrochemical oxygen transfer reaction on synthetic boron doped diamond thin film electrode by Beatrice Marselli (2004), boron-doped diamond electrode may be as a single wafer electrode or may be a coated on a suitable substrate. Preferably, suitable substrates may be selected from but not limited to platinum, stainless steel, graphite, molybdenum, tungsten, titanium, copper, cobalt, chromium, nickel, tantalum, zirconium, niobium, silicon. Further boron-doped diamond electrode can be arranged in mono polar or bi polar mode. Electrolytic cell (50) may be dipped into the apparatus having fitted with conduits for conveying electrolyte solution (20) to pass the electrolyte solution through electrolytic cell (50).

There is at least one means for direct current is provided to energize the electrolytic cell (50). Direct current through means for providing DC power (70) is provided in such a range so that the electrolytic cell (50) is energized completely. Normally, 10 to 2000 amp/m² current density is provided to energize the electrolytic cell but the preferred range is between 100 to 1000 amp/m² which can sufficiently energize the electrolytic cell.

Electrolyte solution in a required quantity may be prepared in an electrolyte solution reservoir (10) which may be passed through filter (30) using pump (40) through conduits of conveying electrolyte solution (20). Further the solution may be passed through energized electrolytic cell (50) through conduits for conveying electrolyte solution (20) to generate in-situ mixed oxidants within the electrolyte solution.

At least one washing apparatus (60) is provided for the purpose of washing, cleaning, disinfecting and sanitizing laundry. Electrolyte solution containing mixed oxidants is supplied to the washing apparatus (60) for washing, cleaning, disinfecting and sanitizing laundry. Washing apparatus (60) is selected from the washing apparatus commercially available. Washing apparatus for the purpose of batch size or continuous process for washing, cleaning, disinfecting and sanitizing laundry may be operated automatically, manually, mechanically.

There is also a plurality of conduits for conveying electrolyte solution (20) may be provided in the system. According to the present invention, at least one electrolyte solution reservoir (10) is connected with at least one filter (30) or at least one pump through conduits, further at least one pump (40) or at least one filter (30) is connected with at least one electrolytic cell (50) having at least one boron-doped diamond electrode through conduits for conveying electrolyte solution. Conduits for conveying electrolyte solution (20) may further extended to the washing apparatus (60) through which electrolyte solution may be supplied to the washing apparatus (60). Conduits for conveying electrolyte solution (20) may be selected from commercially available non-corrosive conduits.

FIG. 2 is a schematic diagram of a system for washing, cleaning, disinfecting and sanitizing laundry using more than one washing apparatus. As per the system disclosed in FIG. 2, more than one washing apparatus (60) can be connected in place of one washing apparatus (60) of FIG. 1. Further washing, cleaning, disinfecting and sanitizing laundry in more than one washing apparatus (60) may be done parallel or simultaneously.

The present invention also encompasses a system and a method for recycling and reuse of electrolyte solution for the purpose of washing, cleaning, disinfecting and sanitizing laundry.

Now, referring to FIG. 3, at least one washing apparatus (60) is connected to at least one reservoir (10) through conduits for conveying electrolyte solution (20). At least one filter (30) is provided between washing apparatus (60) and electrolyte solution reservoir (10) to remove dirt or other unwanted particles from the electrolyte solution. Electrolyte solution of the washing apparatus, after washing and cleaning laundry, may be filtered through filter (30) and further conveyed and stored to the electrolyte solution reservoir (10). The recovered electrolyte solution collected from the washing apparatus may further reused as per the system provided of FIG. 1 or FIG. 2.

The present invention has also disclosed a method for washing, cleaning, disinfecting and sanitizing laundry using the system described hereinabove. A method for washing, cleaning, disinfecting and sanitizing laundry, comprising of:
preparing electrolyte solution in at least one electrolyte solution reservoir comprising water and at least one inorganic salt selected from sodium chloride (NaCl), sodium carbonate (Na₂CO₃), sodium sulfate (Na₂SO₄); potassium chloride (KCl), potassium carbonate (K₂CO₃), ammonium chloride (NH₄Cl), ammonium Sulfate (NH₄)₂SO₄, ammonium carbonate (NH₄)₂CO₃, sodium percarbonate (Na₂CO₃:1.5H₂O₂), potassium percarbonate (K₂CO₃:1.5H₂O₂), ammonium percarbonate or sodium perborate (Na₂BO₃); providing direct current to energize at least one electrolytic cell having at least one boron-doped diamond electrode;
delivering using at least one pump the electrolyte solution filtered through at least one filter to at least one electrolytic cell having at least one boron-doped diamond electrode;
reacting the electrolyte solution electrochemically using at least one energized electrolyte cell having at least one boron-doped diamond electrode to generate mixed oxidants within the electrolyte solution;
supplying to at least one washing apparatus through at least one conduit the electrolyte solution containing in-situ generated mixed oxidants;
operating the washing apparatus for washing, cleaning, disinfecting and sanitizing laundry.

According to the present invention and a system disclosed in the present invention, reservoir (10) is used for the purpose of preparing, storing and conveying electrolyte solution. Electrolyte solution is prepared by dissolving inorganic salt into the water. Inorganic salt is selected from sodium chloride (NaCl), sodium carbonate (Na₂CO₃), sodium sulfate (Na₂SO₄); potassium chloride (KCl), potassium carbonate (K₂CO₃), ammonium chloride (NH₄Cl), ammonium Sulfate (NH₄)₂SO₄, ammonium carbonate (NH₄)₂CO₃, sodium percarbonate (Na₂CO₃:1.5H₂O₂), potassium percarbonate (K₂CO₃:1.5H₂O₂), ammonium percarbonate or sodium perborate (Na₂BO₃). Electrolyte solution using inorganic salt in water is prepared such that the OH radicals may be produced in a sufficient quantity which further able to generate mixed oxidants in a quantity sufficient for washing, cleaning or disinfecting laundry. To prepare electrolyte solution, at least one inorganic salt selected from sodium chloride (NaCl), sodium carbonate (Na₂CO₃), sodium sulfate (Na₂SO₄); potassium chloride (KCl), potassium carbonate (K₂CO₃), ammonium chloride (NH₄Cl), ammonium Sulfate (NH₄)₂SO₄, ammonium carbonate (NH₄)₂CO₃, sodium percarbonate (Na₂CO₃:1.5H₂O₂), potassium percarbonate (K₂CO₃:1.5H₂O₂), ammonium percarbonate or sodium perborate (Na₂BO₃) is dissolved in water in a quantity required for the generation of OH radicals. The ratio of inorganic salt in water may be between 0.5 gm to 10 gm of inorganic salt per liter water but the preferred concentration ratio between 3 gm to 8 gm of inorganic salt per liter water is beneficial to obtain desire results.

pH of the electrolyte solution throughout the process, may be kept between 6.5 to 10.5. pH of electrolyte solution between 7 to 8.5 may provide better conductivity for DC power and thus help to energize the electrolytic cell through which mixed oxidants can be generated in a quantity sufficient for washing, cleaning, disinfecting and sanitizing laundry. Temperature of the process for washing, cleaning, disinfecting and sanitizing laundry may be kept below 50° C. Preferably the temperature between 10° C. to 37° C. during the whole process can provide better affinity towards the generation of OH radicals through which in-situ mixed oxidants can be generated in a quantity sufficient for washing, cleaning, disinfecting and sanitizing laundry.

A filter (30) is provided optionally in between electrolyte solution reservoir (10) and pump (40). Filter (30) can block unwanted particles from the electrolyte solution. If unwanted particle remains in the electrolyte solution then the efficiency of the production of OH radicals may decrease which further leads to the inefficiency of the process for washing, cleaning, disinfecting and sanitizing laundry. Pump (40) can also be fitted before the filter (30) of the system which can help to push electrolyte solution through filter (30). Filter (30) of the system can be selected from the range of the filters which are commercially available but the preferred filter to block unwanted particles, preferably filter with less than 50 microns can give desire results.

Electrolyte solution, after getting it filtered, with the help of pump (40) and conduits (20) for conveying electrolyte solution fitted as per the system, reaches to the electrolytic cell (50).

Direct current through the means for providing DC power (70) is provided in such a range so that the electrolytic cell (50) is energized completely. Normally, 10 to 2000 amp/m² current density is provided to energize the electrolytic cell but the preferred range is between 100 to 1000 amp/m² which can sufficiently energize the electrolytic cell.

Electrolyte solution is passed through the said energized electrolyte cell (50). Electrolyte solution is reacted with energized electrolyte cell (50) electrochemically to produce OH radicals which may further leads to the generation of in-situ mixed oxidants within electrolyte solution. Flow of passing of electrolyte solution through energized electrolytic cell may be as per number of electrolytic cell having boron-doped diamond electrode used in a system for washing, cleaning, disinfecting and sanitizing laundry and the flow may be kept such that the generation of mixed oxidants would be in a quantity required for washing, cleaning, disinfecting and sanitizing laundry. Normally, the flow rate of passing of electrolyte solution through the energized electrolytic cell for at least one electrolytic cell having at least one boron-doped diamond electrode or for the plurality of electrolytic cell having plurality of boron-doped diamond electrode used in a system for washing, cleaning, disinfecting and sanitizing laundry may be between 2,500 liter to 75,000 liter per hour.

In-situ generated mixed oxidants may comprise at least two oxidants selected from ozone, hypochlorite, percarbonate, persulfate and hydrogen peroxide and may vary according to the preparation of electrolyte solution. If the electrolyte solution is prepared by dissolving sodium chloride in water then the mixed oxidants may comprise ozone, perchlorite, hydrogen peroxide and free chlorine. If the electrolyte solution is prepared by dissolving sodium carbonate in water then the mixed oxidants may comprise ozone, percarbonate. If the electrolyte solution is prepared by dissolving sodium chloride and sodium carbonate as a mixture in water then the mixed oxidants may comprise ozone, perchlorite, hydrogen peroxide, percarbonate. Further if the electrolyte solution is prepared using sodium sulfate then the mixed oxidants may comprise ozone, persulfate.

At least one electrolytic cell (50) may be selected from the electrolytic cells having at least one boron-doped diamond electrode. Boron-doped diamond electrode in plurality can also be used according to the application of washing, cleaning, disinfecting and sanitizing laundry. Boron-doped diamond electrodes exhibit interesting electrochemical properties, which include a wide potential window, stability in electrolyte solution, low background current density and high resistance against chemical and electrochemical corrosion. These make the boron-doped diamond electrode a preferred electrode for various electrochemical applications. According to the disclosure of the patents U.S. Pat. No. 5,399,247, U.S. 20060261349 and U.S. Pat. No. 7,407,566 and disclosure in the prior art “Electrochemical oxygen transfer reaction on synthetic boron doped diamond thin film electrode” by Beatrice Marselli (2004), boron-doped diamond electrode may be as a single wafer electrode or may be a coated on a suitable substrate. Preferably, suitable substrates may be selected from but not limited to platinum, stainless steel, graphite, molybdenum, tungsten, titanium, copper, cobalt, chromium, nickel, tantalum, zirconium, niobium, silicon. Further boron-doped diamond electrode can be arranged in mono polar or bi polar mode.

Mixed oxidants generated by the above method having synergistic effect on laundry and give the results above expectation. The results before and after washing, cleaning, disinfecting and sanitizing laundry is given in the best mode of the working of the invention. Generally, all the oxidants play a role for washing, cleaning, disinfecting and sanitizing laundry. The quantity of sufficient quantity of the mixed oxidants can be analyzed by the analysis of ozone quantity of mixed oxidants. The desired quantity of ozone in the mixed oxidants for washing, cleaning, disinfecting and sanitizing should be between 1.5 mg to 20 mg per liter electrolyte solution. The preferred quantity of ozone should be between 3 mg to 15 mg of ozone per liter electrolyte solution to get desire results in washing, cleaning, disinfecting and sanitizing laundry.

Electrolyte solution containing mixed oxidants is then supplied to the washing apparatus (50) for washing, cleaning, disinfecting and sanitizing laundry. Washing apparatus (60) is selected from the washing apparatus commercially available. Washing apparatus (60) for the purpose of batch size or continuous process for washing, cleaning, disinfecting and sanitizing laundry which is operated automatically, manually, mechanically may be used for the purpose of washing and cleaning, disinfecting and sanitizing laundry.

The present invention also relates to recycling and reuse of electrolyte solution containing mixed oxidants recovered from the method described herein above. The electrolyte solution may contain dirt and other unwanted particles that can decrease the efficiency of the electrolyte solution. Recovered electrolyte solution may be used for further washing, cleaning, disinfecting and sanitizing laundry only after removal of dirt and unwanted particles. The recovered electrolyte solution of the washing apparatus after washing and cleaning laundry is passed through filter (30) and conveyed to the electrolyte solution reservoir (10). The electrolyte solution may be further recycled and reused for washing, cleaning, disinfecting and sanitizing laundry using the method described hereinabove.

Now, the methods for washing, cleaning, disinfecting and sanitizing laundry and their results, without any limitation may be described as follows:

### EXAMPLE—1

### A Method For Washing And Cleaning of Laundry

(1) Required conditions:
Electrolytic cell having at least one boron-doped diamond electrode arranged in bi polar mode
pH of electrolyte solution: 7
Temperature: 25° C.-30° C.
Current density of direct current: 100 amp/m²

(2) Soiling method and parameter:

Soiling is done with following food/sauces on the cotton clothes
(a) Chocolate sauce
(b) Soya sauce
(c) Salsa sauce
(d) Salad spread
(e) Tomato ketchup
(f) Plain cloth for reference

(3) Total weight of cotton clothes: 1330 gm

### Washing And Cleaning Of Laundry

In an electrolyte solution reservoir, Sodium chloride (120 gm) is dissolved in 30 liter water to prepare electrolyte solution. The said electrolyte solution is passed through the filter of 50 microns using pump to block unwanted particles remains present in the electrolyte solution. Direct current having current density of 100 amp/m² for 15 minutes is provided to the electrolytic cell having boron-doped diamond electrode to energize the electrolytic cell. Electrolyte solution after filtering it, is thus passed through the energized electrolytic cell having at lest one boron-doped diamond electrode. Passing of electrolyte solution through the electrolytic cell leads to the electrochemical reaction to generate mixed oxidants containing ozone, hypochlorite, hydrogen peroxide in which ozone concentration is 2.4 ppm in electrolyte solution. During the whole process, temperature 25° C.-30° C. was maintained. The said electrolyte solution containing mixed oxidants is supplied to the top loaded washing machine wherein cotton clothes (1330 gm) are kept for washing and cleaning purpose. Washing for 20 minutes, and spinning for 10 minutes in washing machine at a temperature between 25° C.-30° C. without any addition of soap, powder to give following results:

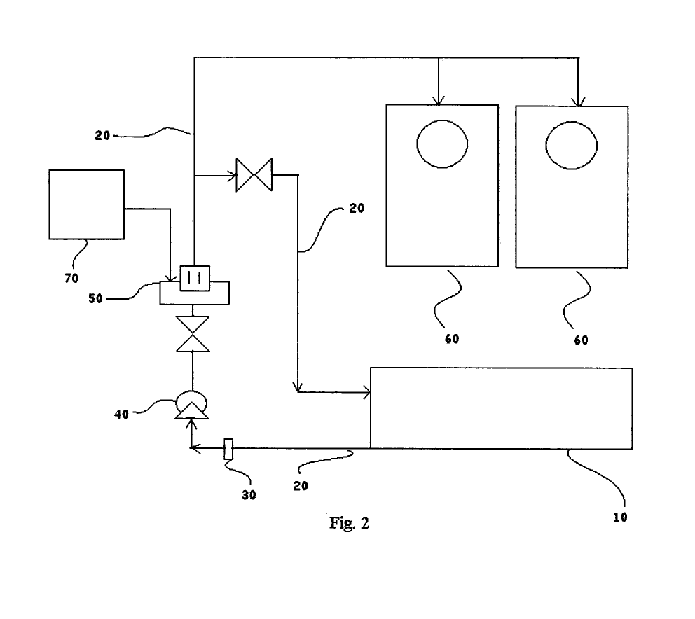

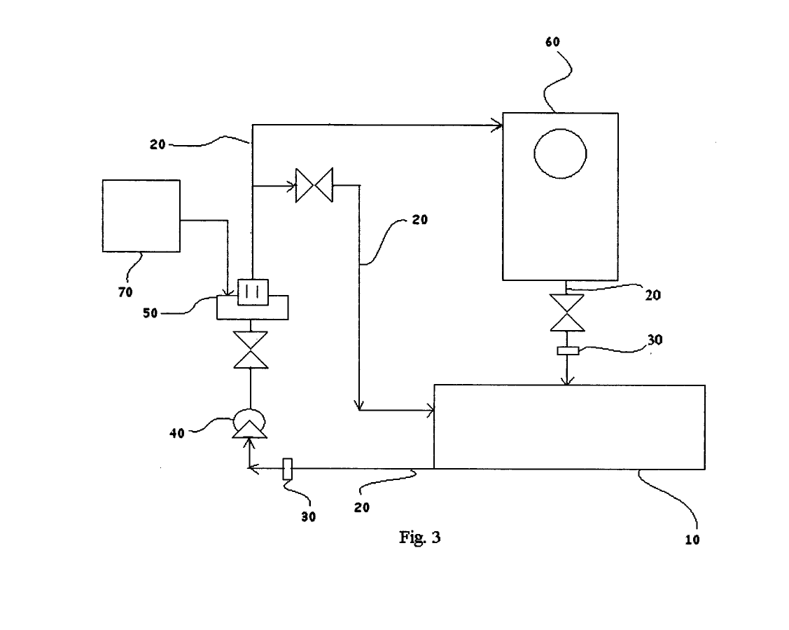

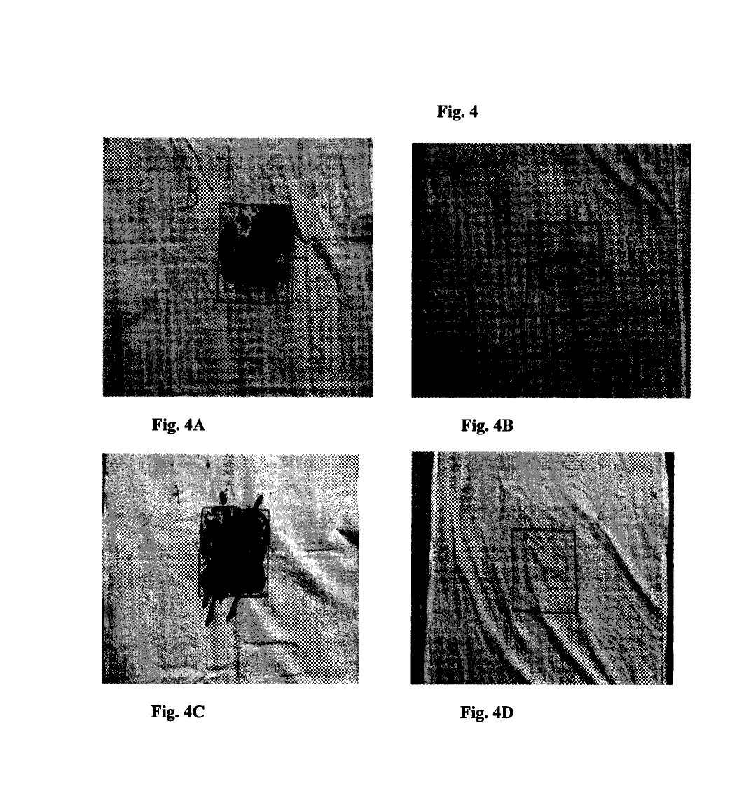

Results: Results before and after washing and cleaning laundry are shown in FIG. 4. Clothes got completely washed and cleaned in first wash cycle.

### EXAMPLE—2

### A Method For Washing And Cleaning of Laundry

(1) Required conditions:
Electrolytic cell having at least one boron-doped diamond electrode
pH of electrolyte solution: 7.5
Temperature: 33° C.-37° C.
Current density of direct current: 100 amp/m²

(2) Soiling method and parameter:

Soiling is done with following food/sauces on the cotton clothes
(a) Chocolate sauce
(b) Soya sauce
(c) Salsa sauce
(d) turmeric
(e) Tomato ketchup
(f) Plain cloth for reference
(g) Blood

(3) Total weight of cotton clothes: 1000 gm

(4) Types of clothes: cotton & Terry cotton

### Washing And Cleaning Of Laundry

In an electrolyte solution reservoir, NaHCO₃ (120 gm) is dissolved in 30 liter water to prepare electrolyte solution. The said electrolyte solution is passed through the filter of 50 microns using pump to block unwanted particles remains present in the electrolyte solution. Direct current having current density of 100 amp/m² is provided to the electrolytic cell having boron-doped diamond electrode to energize the electrolytic cell. Electrolyte solution after filtering it, is thus passed through the energized electrolytic cell having at lest one boron-doped diamond electrode. Passing of electrolyte solution through the electrolytic cell leads to the electrochemical reaction to generate mixed oxidants containing ozone, percarbonate in which ozone concentration is 2.4 ppm in electrolyte solution. During the whole process, temperature 33° C.-37° C. is maintained. The said electrolyte solution containing mixed oxidants is supplied to the top loaded washing machine wherein clothes (1000 gm) are kept for washing and cleaning purpose. Washing for 20 minutes, and spinning for 10 minutes in washing machine at a temperature between 33° C.-37° C. without any addition of soap, powder to give following results:

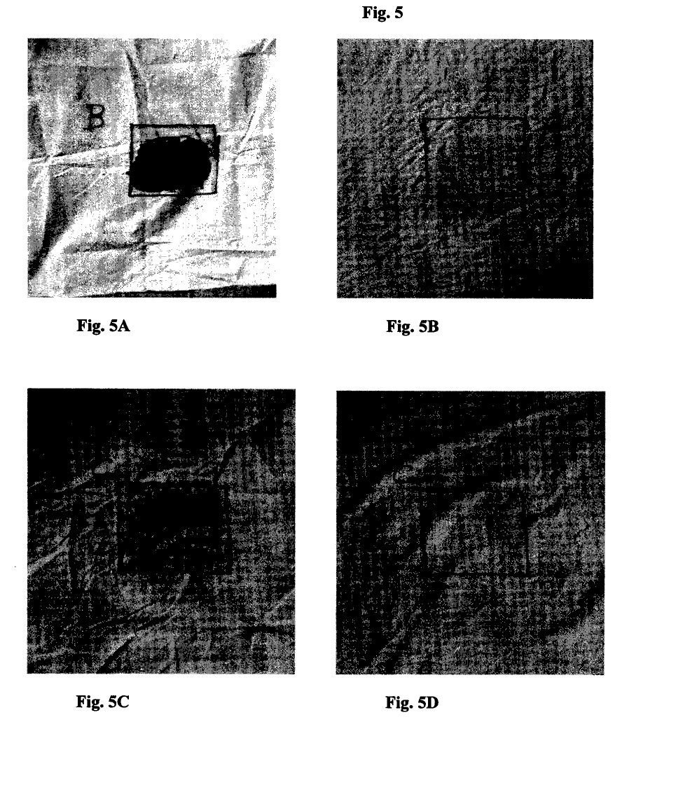

Results: Results before and after washing and cleaning laundry are shown in FIG. 5. Clothes got completely washed in first wash cycle. Stains are completely removed in second wash.

### EXAMPLE—3

### A Method For Disinfecting And Sanitizing Laundry

(1) Required conditions:
Electrolytic cell having at least one boron-doped diamond electrode
pH of electrolyte solution: 7.9
Temperature: 25° C.-28° C.
Current density of direct current: 100 amp/m²

(2) Total weight of clothes: 6 gm

### Method For Disinfecting And Sanitizing Laundry

In an electrolyte solution reservoir, NaCl (40 gm) is dissolved in 10 liter water to prepare electrolyte solution. The said electrolyte solution is passed through the filter of 50 microns using pump to block unwanted particles remains present in the electrolyte solution. Direct current having current density of 100 amp/m² is provided to the electrolytic cell having boron-doped diamond electrode to energize the electrolytic cell. Electrolyte solution after filtering it, is thus passed through the energized electrolytic cell having at lest one boron-doped diamond electrode. Passing of electrolyte solution through the electrolytic cell leads to the electrochemical reaction to generate mixed oxidants containing ozone, perchloride, hydrogen peroxide in which ozone concentration is 3 ppm in electrolyte solution. During the whole process, temperature 25° C.-28° C. is maintained. Clothes infected with e-coli (initial colony: 10⁶) are treated with electrolyte solution containing mixed oxidants for 10 minutes. Final colony of e-coli bacteria of the clothes reduced up to 0 by the said washing and cleaning method.

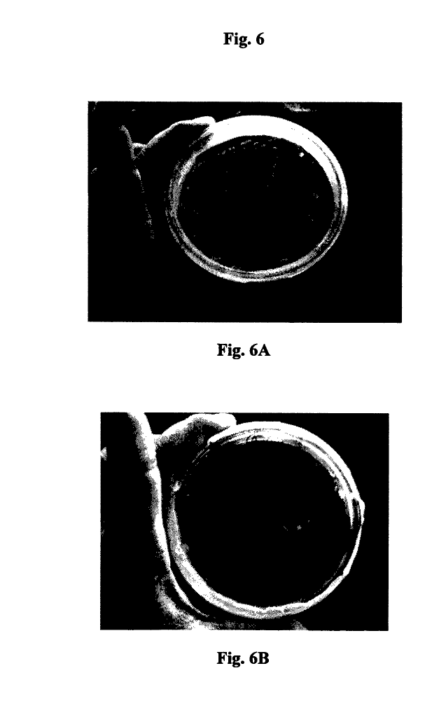

Results: Clothes get disinfected and sanitized completely after its treatment with electrolyte solution containing mixed oxidants. Results are shown in FIG. 6.

| | Organism by | | | Final colony |
|---|---|---|---|---|
| Type of | which specimen | Initial colony | Retention time | after wash |
| specimen | is infected | (CFU) | (min) | (CFU) |
| Clothes | E-Coli | 10⁶ | 10 | 0 |

### EXAMPLE—4

### Industrial Process For Cleaning, Washing, Laundry Using Electrolytic Cell Having Boron-Doped Diamond Electrode

An industrial process with examples without any limitations in a similar way with the required parameters can be shown as follows:

| PARAMETERS OF METHOD | EXAMPLE 1 | EXAMPLE 2 | EXAMPLE 3 | EXAMPLE 4 |
|---|---|---|---|---|
| Types of Clothes & weight of | Towels (36) | Bed sheets (36) | Towels (36) | Napkins (36) |
| clothes (in kg) | | | | |
| Soiling level | High | Normal | Normal | Normal |
| Inorganic | Na₂CO₃ (4) | NaC1 (2) | NaCl (4) | Sodium |
| salt (in Kg) | NaCl (2) | | | perborate (1) |
| | | | | Sodium |
| | | | | percarbonate (1) |
| Quantity | 950 | 950 | 950 | 500 |
| of water (in Liter) | | | | |
| Temperature & pH | 33-35°C. & 10.5 | 24-27°C. & 7.8 | 24-27°C. & 8.0 | 35-37°C. & 10.5 |
| Current density of | 800 | 480 | 650 | 400 |
| direct current (Amp/m²) | | | | |
| Time for energizing the | 20 | 30 | 45 | 40 |
| electrolytic cell (minutes) | | | | |
| Ozone in in-situ generated | 11 | 10 | 12 | 14 |
| mixed oxidants (ppm) | | | | |
| Electrolyte solution containing | 220 | 400 | 370 | 200 |
| mixed oxidants used (liter) | | | | |
| Recycling and reuse of | Recycling and | Recycling and | Recycling and | Recycling and |
| recovered electrolyte solution | reusable | reusable | reusable | reusable |
| Results | Results as per | Results as per | Results as per | Results as per |
| | example-1 | example-1 | example-1 | example-1 |

1. A system for washing, cleaning, disinfecting and sanitizing laundry, comprising:
at least one electrolyte solution reservoir;
at least one filter;
at least one electrolytic cell having at least one boron-doped diamond electrode;
at least one pump;
at least one washing apparatus;
at least one means for providing DC power; and
a plurality of conduits for conveying electrolyte solution.

2. A system as claimed in claim 1 wherein boron-doped diamond electrode is a single wafer electrode.

3. A system as claimed in claim 1 wherein boron-doped diamond electrode is selected from the electrodes coated on a suitable substrate.

4. A system as claimed in claim 3 wherein suitable substrate is selected from the metals preferably from platinum, stainless steel, graphite, molybdenum, tungsten, titanium, copper, cobalt, chromium, nickel, tantalum, zirconium, niobium, silicon.

5. A system as claimed in claim 1 wherein boron-doped diamond electrode is arranged in mono polar or bi polar mode.

6. A system as claimed in claim 1 wherein washing apparatus is selected from the apparatus which is operated electrically, mechanically or manually.

7. A system as claimed in claim 1 further comprising:
providing at least one filter to filter electrolyte solution recovered from washing apparatus
providing means for conveying filtered electrolyte solution to the electrolyte solution reservoir

8. A method for washing, cleaning, disinfecting and sanitizing laundry, comprising:
preparing electrolyte solution in at least one electrolyte solution reservoir comprising water and at least one inorganic salt selected from sodium chloride (NaCl), sodium carbonate (Na₂CO), sodium sulfate (Na₂SO₄); potassium chloride (KCl), potassium carbonate (K₂CO₃), ammonium chloride (NH₄Cl), ammonium Sulfate (NH₄)₂SO₄) ammonium carbonate (NH₄)₂CO₃, sodium percarbonate (Na₂CO₃:1.5H₂O₂),
potassium percarbonate (K₂CO₃:1.5H₂O₂), ammonium percarbonate or Sodium perborate (Na₂BO₃);
providing direct current to energize at least one electrolytic cell having at least one boron-doped diamond electrode;
delivering using at least one pump the electrolyte solution filtered through at least one filter to at least one electrolytic cell having at least one boron-doped diamond electrode;
reacting the electrolyte solution electrochemically using at least one energized electrolyte cell having at least one boron-doped diamond electrode to generate mixed oxidants within the electrolyte solution;
supplying to at least one washing apparatus through at least one conduit the electrolyte solution containing in-situ generated mixed oxidants;
operating the washing apparatus for washing, cleaning, disinfecting and sanitizing laundry.

9. A method as claimed in claim 8 wherein electrolyte solution is prepared in the concentration range of 0.5 to 10.0 gm of inorganic salt per liter of water, preferably in the concentration range of 3 to 8 gm of inorganic salt per liter of water.

10. A method as claimed in claim 8 wherein current density of direct current is provided in a range between 10-2000 amp/m, more preferably between 100-1000 amp/m for electrochemical reaction of electrolyte solution.

11. A method as claimed in claim 8 wherein in-situ generated mixed oxidants comprising at least two oxidants selected from Ozone, hypochlorite, percarbonate, persulfate and hydrogen peroxide produced in the electrolysis of electrolyte solution.

12. A method as claimed in claim 8 wherein the concentration of ozone in the mixed oxidants is between 1.5 to 20 mg per liter of water.

13. A method as claimed in claim 8 wherein boron-doped diamond electrode is a single wafer electrode.

14. A method as claimed in claim 8 wherein boron-doped diamond electrode is selected from the electrodes coated on a suitable substrate.

15. A method as claimed in claim 14 wherein suitable substrate is selected from the metals preferably from platinum, stainless steel, graphite, molybdenum, tungsten, titanium, copper, cobalt, chromium, nickel, tantalum, zirconium, niobium, silicon.

16. A method as claimed in claim 8 wherein boron-doped diamond electrode is arranged in mono polar or bi polar mode.

17. A method as claimed in claim 8 wherein the means of circulating electrolyte solution is selected from the materials which are non-corrosive.

18. A method as claimed in claim 8 wherein washing apparatus is selected from the apparatus which is operated electrically, mechanically or manually.

19. A method as claimed in claim 8 wherein a method is carried out at pH 6.5 to 10.5.

20. A method as claimed in claim 8 wherein a method is carried out at a temperature below 50° C., preferably at a temperature between 10 to 37° C.

21. A method as claimed in claim 8, further comprising:
recovering used electrolyte solution from the washing apparatus in at least one reservoir;
repeating the method claimed in claim 8 for washing, cleaning, disinfecting and sanitizing laundry.

22. A method as claimed in claim 21 wherein a method is carried out at pH 6.5 to 10.5, preferably 7 to 8.5.

23. A method as claimed in claim 21 wherein a method is carried out at a temperature below 50° C., preferably at a temperature between 10 to 37° C.
